(19) United States
(12) Patent Application Publication
(10) Pub. No.: US 2025/0090489 A1
(43) Pub. Date: Mar. 20, 2025

(54) CATIONIC POLYMERIC NANOCARRIERS INHIBIT CHEMOTHERAPY-INDUCED CANCER METASTASIS AND COGNITIVE IMPAIRMENT

(71) Applicant: The Trustees of Columbia University in the City of New York, New York, NY (US)

(72) Inventors: Kam W. LEONG, New York, NY (US); Tianyu LI, New York, NY (US); Tolulope O. AKINADE, New York, NY (US); Divya BHANSALI, New York, NY (US); Hongxia WANG, New York, NY (US)

(21) Appl. No.: 18/970,049

(22) Filed: Dec. 5, 2024

Related U.S. Application Data
(63) Continuation of application No. PCT/US2023/068012, filed on Jun. 6, 2023.
(60) Provisional application No. 63/349,492, filed on Jun. 6, 2022.

(51) Int. Cl. A61K 31/337; A61K 31/704; A61K 47/54; A61K 47/69; A61P 35/04

(52) U.S. Cl. CPC A61K 31/337; A61K 31/704; A61K 47/554; A61K 47/6907; A61P 35/04

(57) ABSTRACT

PAMAM-based nanocarriers with high loading efficiency of chemotherapeutics and high cfNA binding ability enable sustained delivery of chemotherapeutics while inhibiting systemic inflammation caused by the chemotherapy. This is useful for treating both primary and metastatic tumors with attenuated cognitive impairment.

### REFERENCE TO RELATED APPLICATIONS

This application is a continuation of International Application No. PCT/US2023/068012, filed Jun. 6, 2023, entitled “Cationic Polymeric Nanocarriers to Inhibit Chemotherapy-Induced Cancer Metastasis and Cognitive Impairment”, which claims priority to U.S. Provisional Application No. 63/349,492, filed Jun. 6, 2022, entitled “Cationic Polymeric Nanocarriers to Inhibit Chemotherapy-Induced Cancer Metastasis and Cognitive Impairment”. The benefit under 35 USC § 119(e) of the United States provisional application is hereby claimed, and each of the aforementioned applications are hereby incorporated herein by reference in their entireties.

### GOVERNMENT LICENSE RIGHTS

This invention was made with government support under grant no. AR073935 awarded by the National Institutes of Health (NIH), and grant no. W81XWH1910463 awarded by the Department of Defense (DoD). The government has certain rights in the invention.

### BACKGROUND

Cancer is the leading cause of death in most countries. Among females, breast cancer is the most-commonly diagnosed cancer and has the second highest death rate. Many patients with early-stage non-metastatic breast cancer can be cured with a combination of surgery, chemotherapy, and radiotherapy, but roughly 50% of patients develop distant organ metastases, which are typically incurable. Chemotherapeutics such as paclitaxel and doxorubicin are used to treat primary breast cancer in combination with surgery. Although these drugs inhibit primary tumor growth, growing evidence suggests that they may promote metastasis by causing elevated levels of proinflammatory cell-free nucleic acids (cfNAs), which are released by damaged cells into the tumor microenvironment. New studies have started to focus on attenuating the prometastatic effects of chemotherapy.

Cognitive impairment is another side effect that cancer patients who have had chemotherapy may face. These cognitive impairments are also termed chemobrain, chemofog, or chemotherapy-induced cognitive impairment. Chemobrain was first cited in 1980 when a cohort of cancer patients scored significantly lower in cognitive function assessments during and after chemotherapy, and it was advised that psychiatrists be aware of chemotherapy as a possible source of behavioral changes. In qualitative studies, breast cancer survivors reported problems with concentration, working memory, and multi-tasking, pointing to changes in executive function and learning/memory. Cohort studies found that chemotherapy-induced cognitive impairment could persist anywhere from 4-10 years with subjects displaying parahippocampal gyrus hyporesponsiveness during a paired associate task for assessing episodic memory. Therefore, developing novel nanomedicine to mitigate chemobrain is highly desired.

Elevated levels of cfNAs-single- and double-stranded DNA and RNA-surrounding tumors and in circulation following chemotherapy have been associated with cancer metastasis and chemobrain. cfNAs are damage-associated molecular patterns (DAMPs) that induce chronic inflammation by activating toll-like receptors (TLRs); for example, dsRNA activates TLR3, ssRNA activates TLR8, and ssDNA activates TLR9. Activated TLRs upregulate MYD88 and NF-κB to induce secretion of inflammatory cytokines, and TLR-induced systemic inflammation appears to promote tumor metastasis and cognitive impairment. cfNAs are being explored as biomarkers for cancer diagnosis and prognosis but are seldom recognized as therapeutic targets for preventing metastasis and chemobrain.

Recently, we discovered that cationic polyamidoamine (PAMAM) dendrimers can scavenge nucleic acids via electrostatic interactions. When used to treat inflammatory and autoimmune diseases, PAMAM scavenges cfNAs, inhibiting cfNA-induced TLR activation and TLR-induced inflammation. In pancreatic cancer and breast cancer models, PAMAM dendrimers inhibit metastasis by scavenging cfNAs. With careful molecular design based on PAMAM dendrimers, cationic polymeric nanocarriers for chemotherapeutics can inhibit both cancer metastasis and cognitive impairment induced by chemotherapy.

### SUMMARY

We developed cationic polymer-based nanoparticles capable of encapsulating chemotherapy and binding to inflammatory circulating cell-free DNA molecules. This nanoparticle technology has been found to reduce chemotherapy-induced systemic inflammation and cancer metastasis in vivo. These nanoparticles can thereby reduce likely neuroinflammation by reducing chemotherapy-related systemic inflammation and thereby mitigate chemobrain symptoms. Cognitive impairment is one of the side effects that cancer patients who have had chemotherapy may face. These cognitive impairments are termed chemobrain, chemofog or chemotherapy-induced cognitive impairment. In qualitative studies, cancer survivors have reported problems with concentration, anxiety, working memory, and multi-tasking, pointing to changes in executive function and learning/memory.

Chemotherapy promotes chronic inflammation via necrotic cancer cell death and the release of their cellular contents, termed damage-associated molecular patterns (DAMPs), into the blood. DAMPs include cell-free nucleic acids (such as cell-free DNA and miRNA) which can activate immune toll-like receptors (TLRs). The persistence of DAMPs in blood induces a state of chronic inflammation with elevated inflammatory cytokine levels in circulation which can then enter the central nervous system and induce neuroinflammation. Neuroinflammation has been identified as a risk factor in the pathophysiology of depression, and a positive correlation between chemobrain and depression has been found. Chemotherapy is a mainstay of cancer treatment, so it is imperative to find methods that can minimize off-target inflammation and inadvertent cognitive impairment. Due to the fact that our nanomaterials can reduce the systemic inflammation induced as a byproduct of chemotherapy treatment, they may also mitigate the cognitive effects that result from chemotherapy treatment by reducing systemic inflammation and thereby reducing neuro-inflammation.

In some aspects, the disclosure concerns polymeric nanocarriers for delivery of chemotherapeutics comprising cationic polyamidoamine (PAMAM) dendrimers modified with cholesterol. Some polymeric nanocarriers additionally comprise a chemotherapy drug. In some embodiments, the chemotherapy drug is one or both of paclitaxel or doxorubicin.

In certain embodiments, the PAMAM (polyamidoamine) dendrimers modified with cholesterol comprise at least one cholesterol residue at a terminal position of the PAMAM. In certain embodiments, the cholesterol residue is attached to an external amine functional group of the PAMAM.

Some polymeric nanocarriers have a sphere-like shape.

Other aspects of the disclosure concern methods of treating cancer in a patient by administering a polymeric nanocarrier disclosed herein to a patient. In some embodiments, the cancer is one or both of a primary and metastatic tumor.

In certain embodiments, the method prevents or reduces chemotherapy-induced cognitive impairment in the patient. In some embodiments, the method prevents or reduces prometastatic effects of chemotherapy in the patient. The cationic polyamidoamine (PAMAM) dendrimers modified with cholesterol was also found to reduce cell free nucleic acid (cfNA) levels within the patient.

In certain embodiments, the nanocarriers and methods disclosed herein are used for the treatment of cancer. In some embodiments, the nanocarriers and methods are utilized after surgery, chemotherapy or radiotherapy to prevent cancer relapse.

In certain embodiments, the polymeric nanocarrier delivers at least one immune check-point inhibitor to the patient.

In yet another aspect, the disclosure includes methods of making the polymeric nanocarriers for delivery of chemotherapeutics described herein. Some methods comprise utilizing an oil-in-water emulsion process to produce a nanocarrier comprising cationic polyamidoamine (PAMAM) dendrimers modified with cholesterol and a chemotherapy drug.

### BRIEF DESCRIPTION OF THE FIGURES

FIGS. 1A-1F illustrate synthesis and characterizations of cationic nanocarriers. 1A) Synthesis route of cholesterol modified PAMAM dendrimers; 1B) TEM images of cationic nanocarrier PAMAM-Chol(5); 1C) Basis physicochemical properties of cationic nanocarriers; 1D) 1H-NMR spectra of the polymers; 1E) DNA binding efficiency of cationic nanocarriers; 1F) Cytotoxicity of cationic nanocarriers to MDA-MB-231 cells.

FIGS. 2A-2K illustrate anticancer efficacy of cationic nanoparticles. 2A) Schematic illustration of the NSG mouse study; 2B) Primary tumor growth curve; 2C) Tumor images; 2D) In vivo imaging of tumor metastasis; 2E) Quantification of in vivo imaging signals; 2F) Ex vivo imaging of lungs showing tumor metastasis; 2G) Quantification of ex vivo imaging signals; 2H) Probability maps created by machine learning based on hematoxylin and eosin (H&E) staining lung sections showing lung metastasis; 21) Quantification of metastasis levels by machine learning; 2J) Serum cell free DNA (cfDNA) levels at different time points; 2K) Serum cfDNA levels at the final time point.

FIGS. 3A-3C illustrate evaluation of cognitive impairment. 3A) Schematic illustration of the Balb/c mouse study; 3B) Total travel distance of mice in open field tests; 3C) Distance traveled in center area by mice during open field tests.

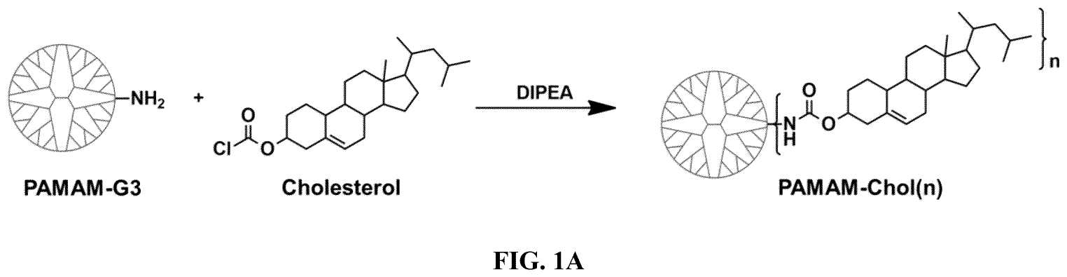

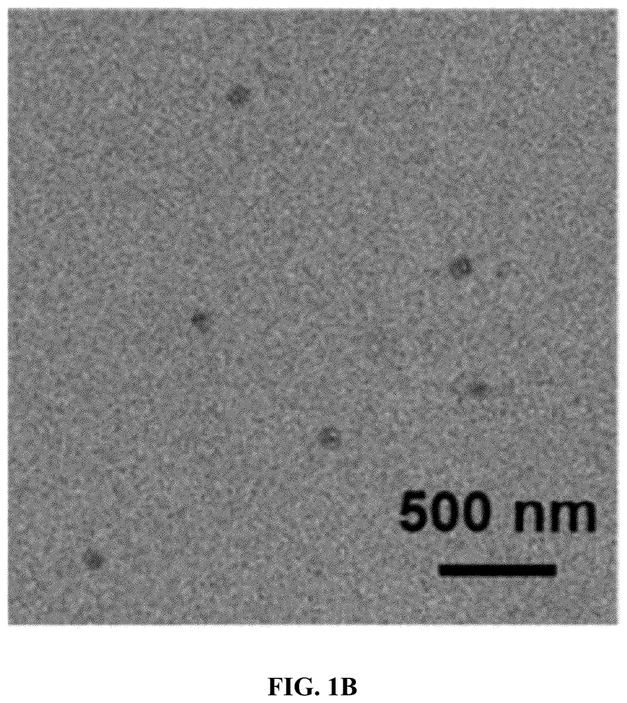

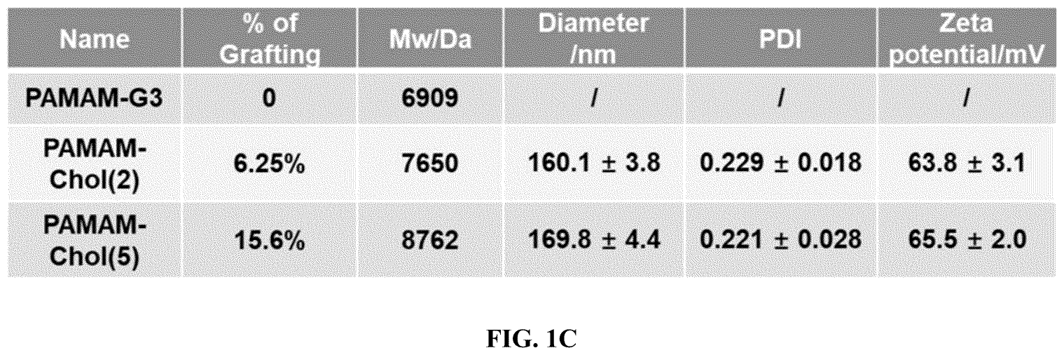

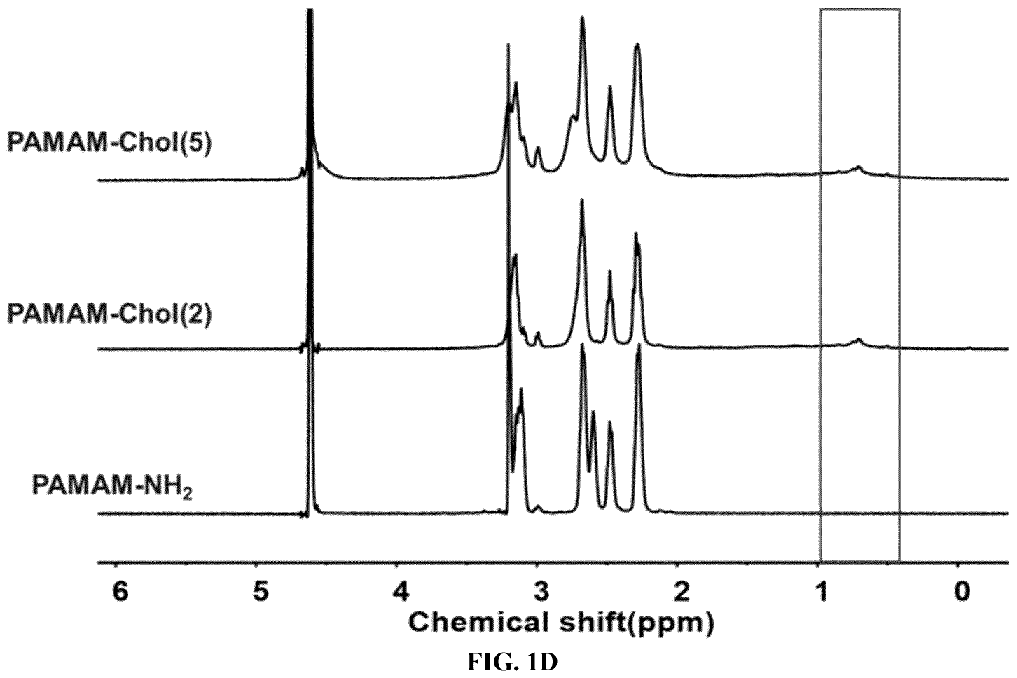

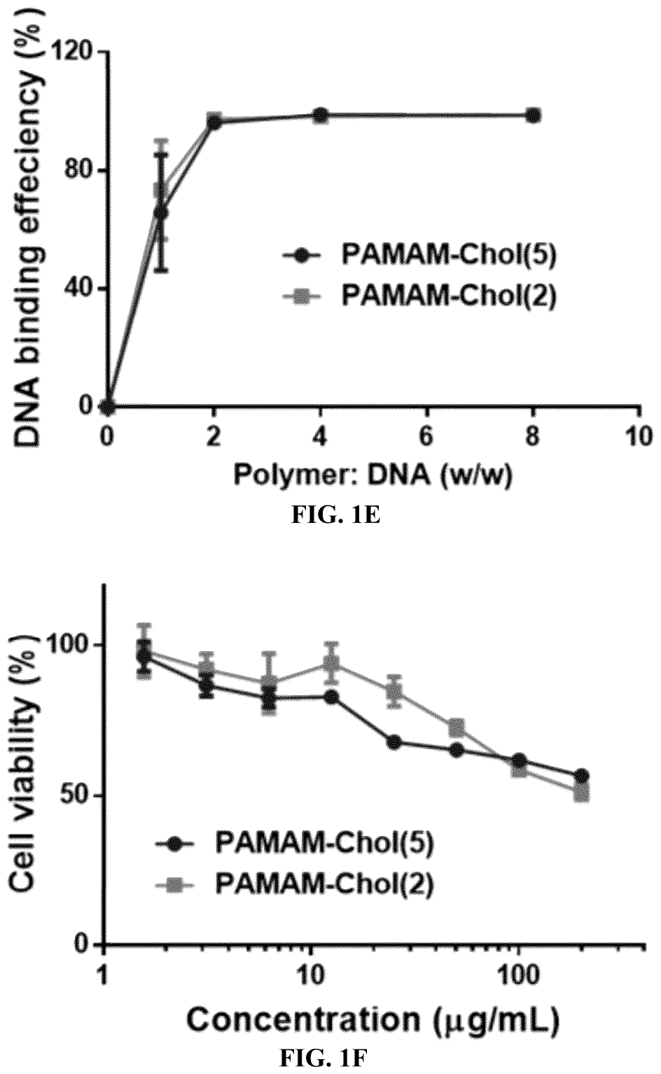

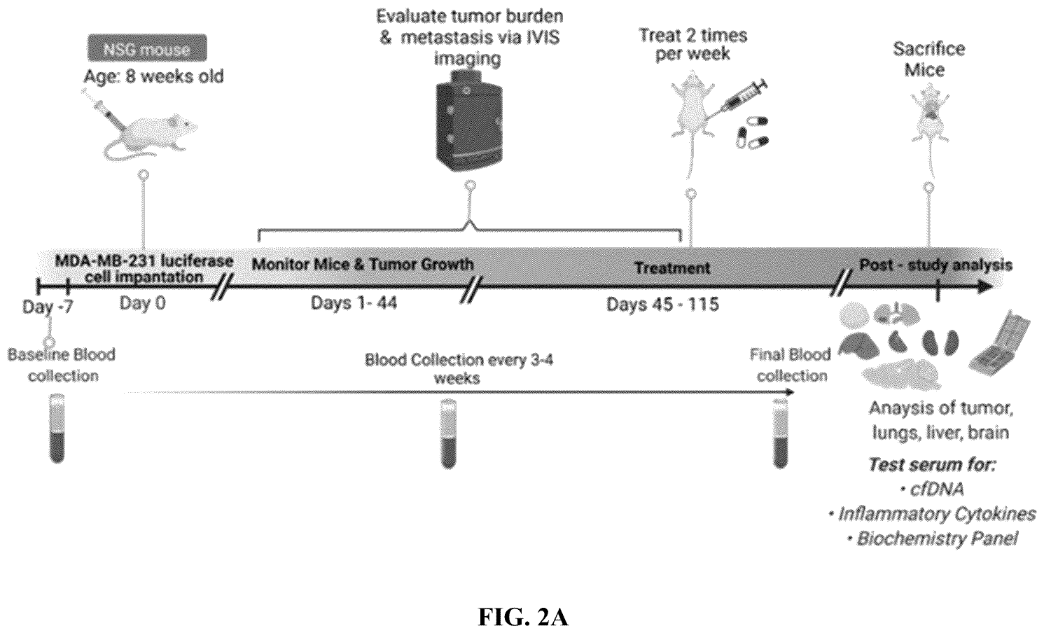

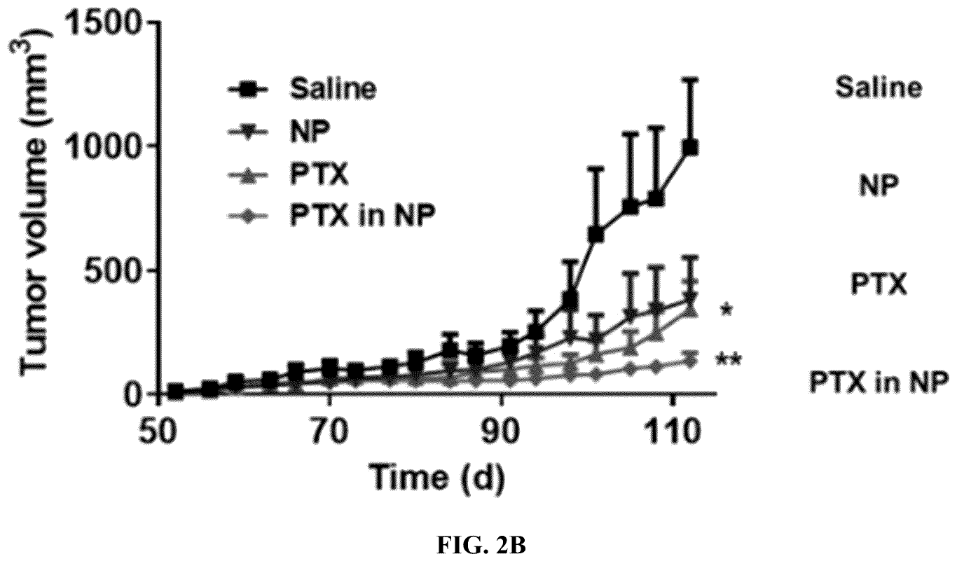

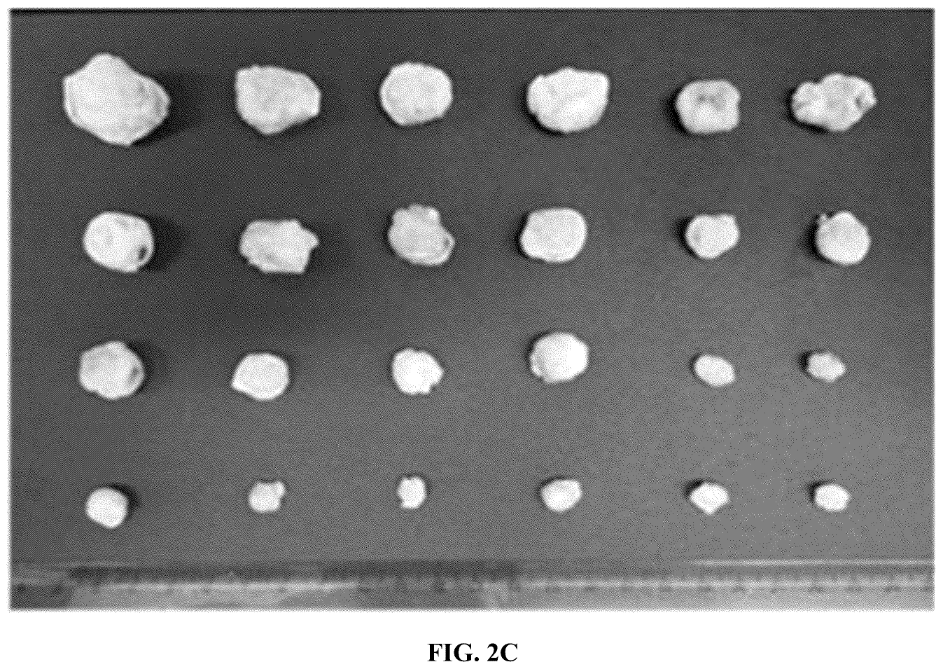

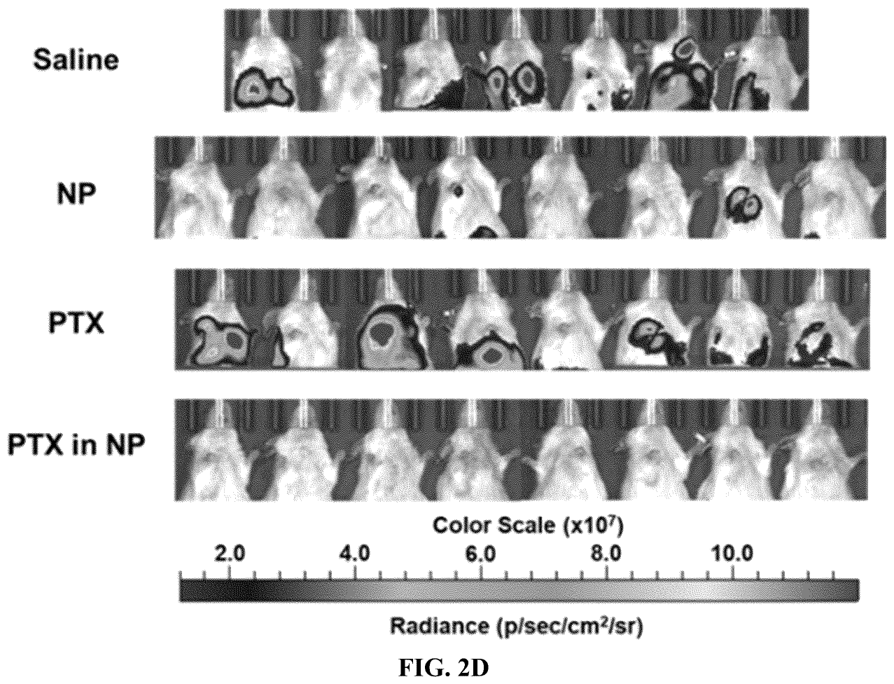

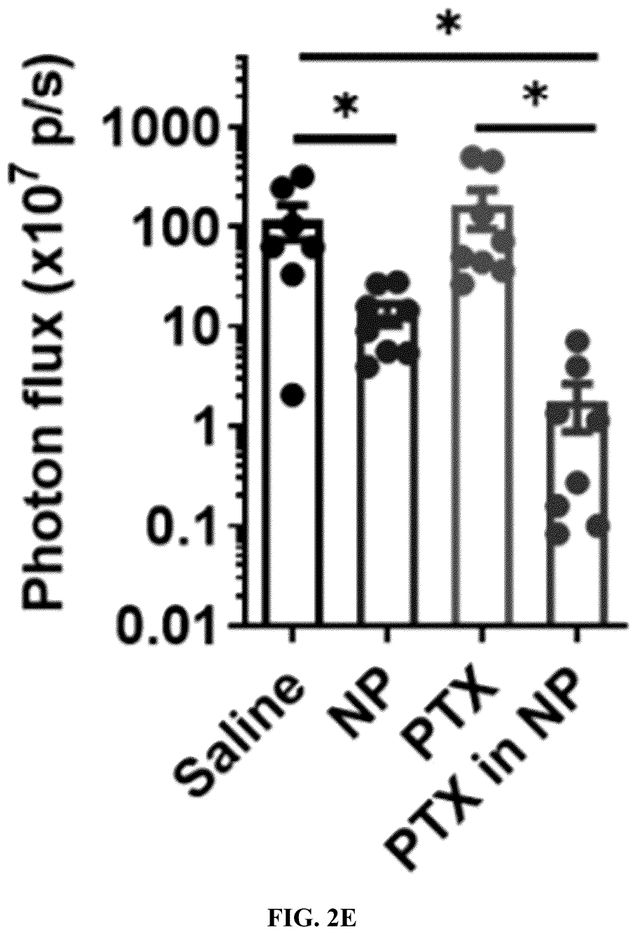

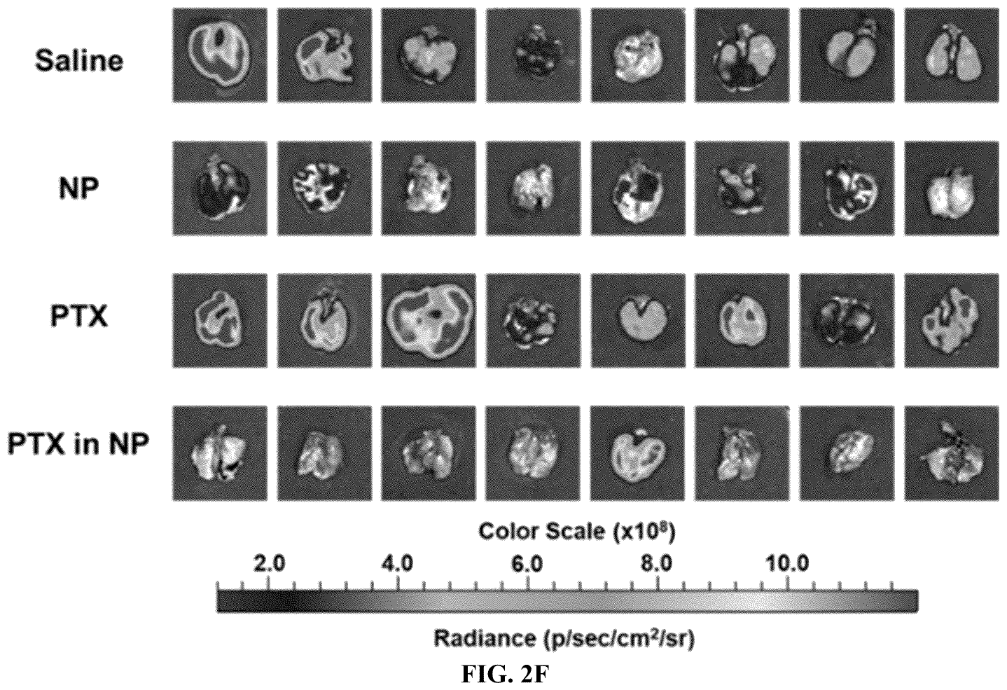

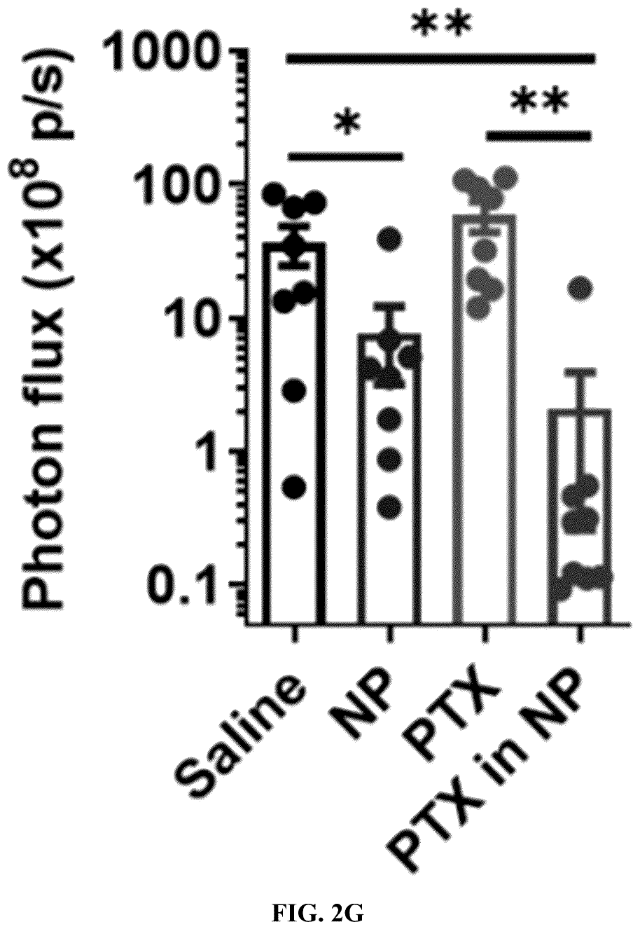

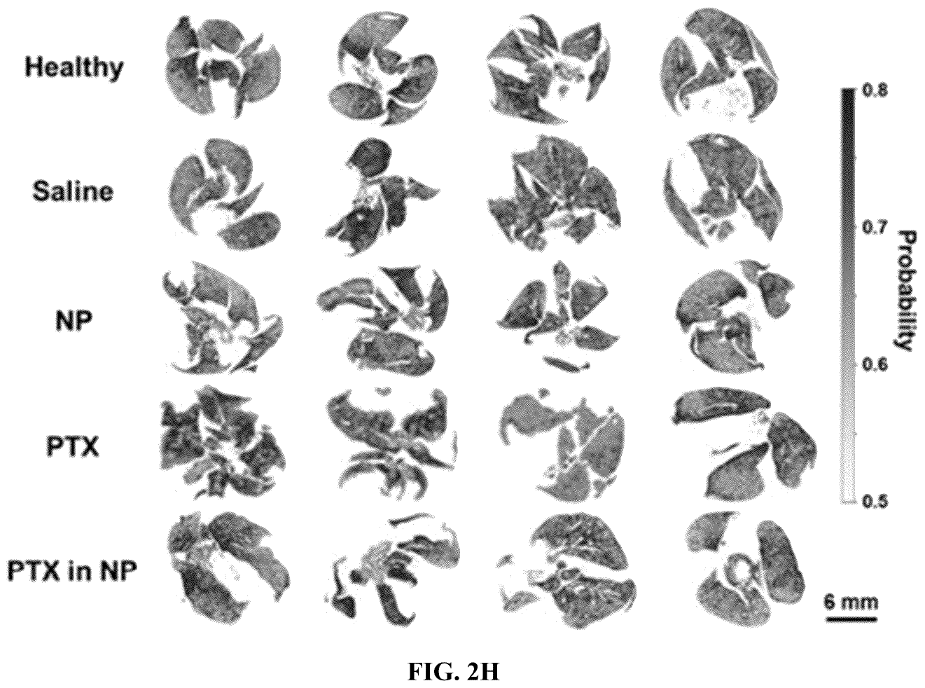

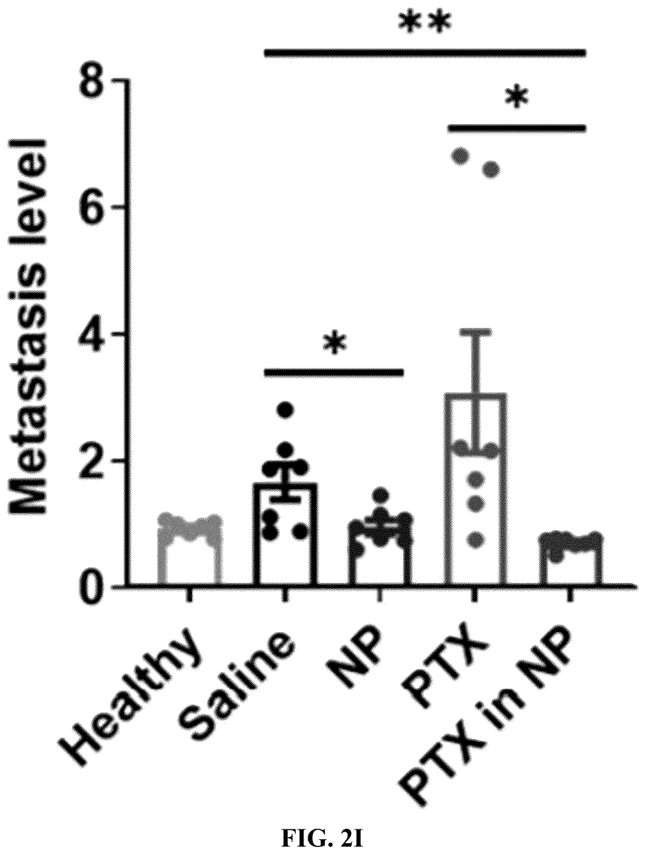

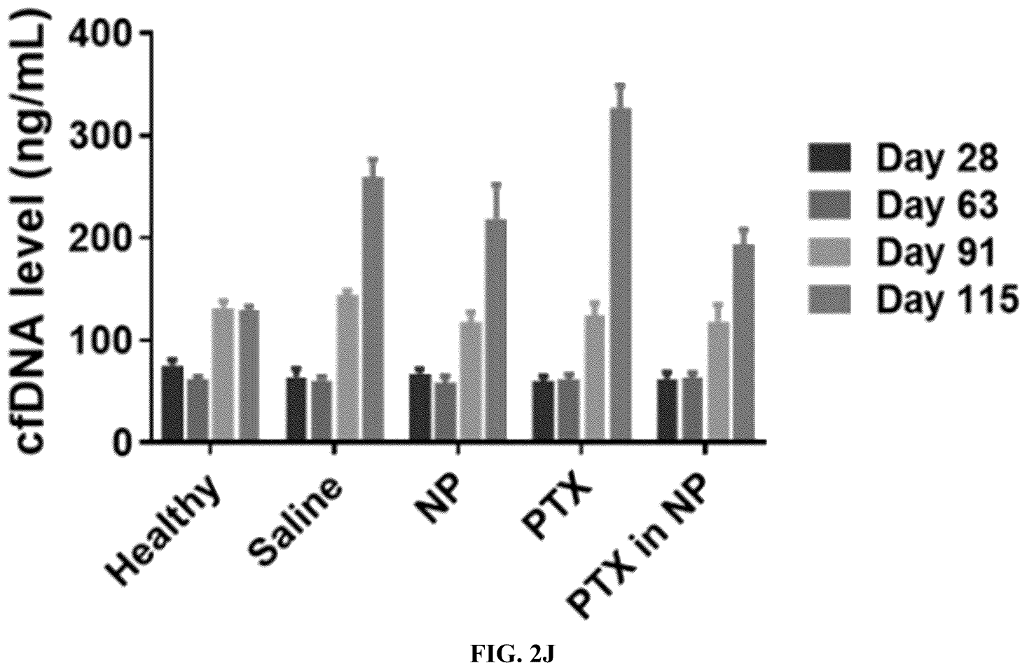

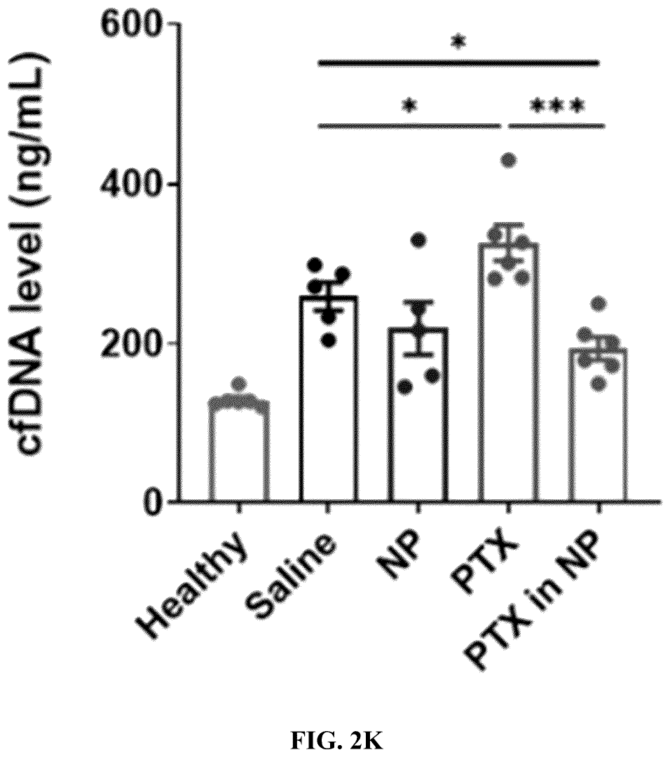

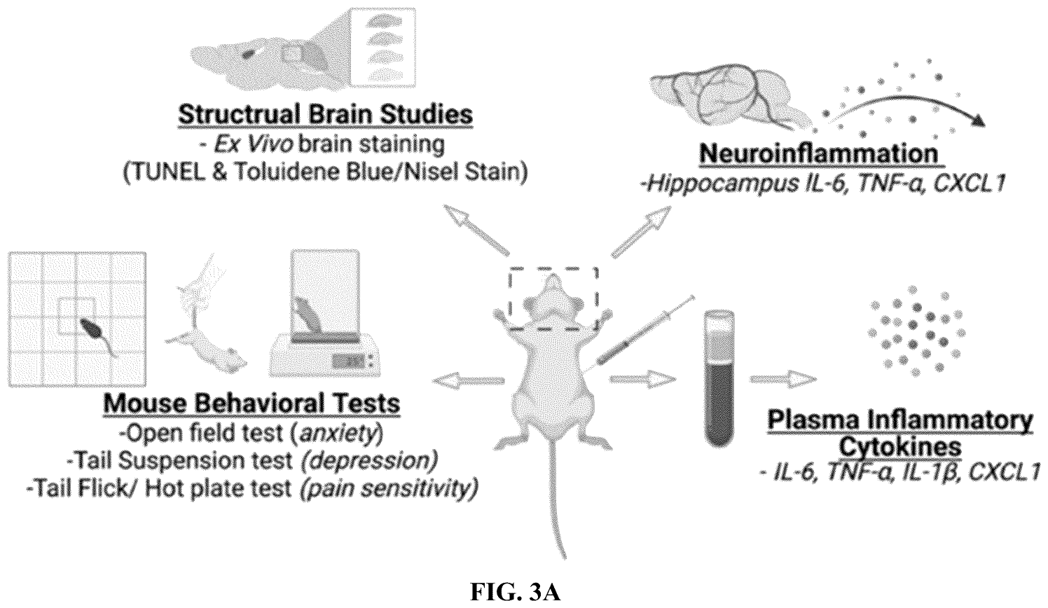

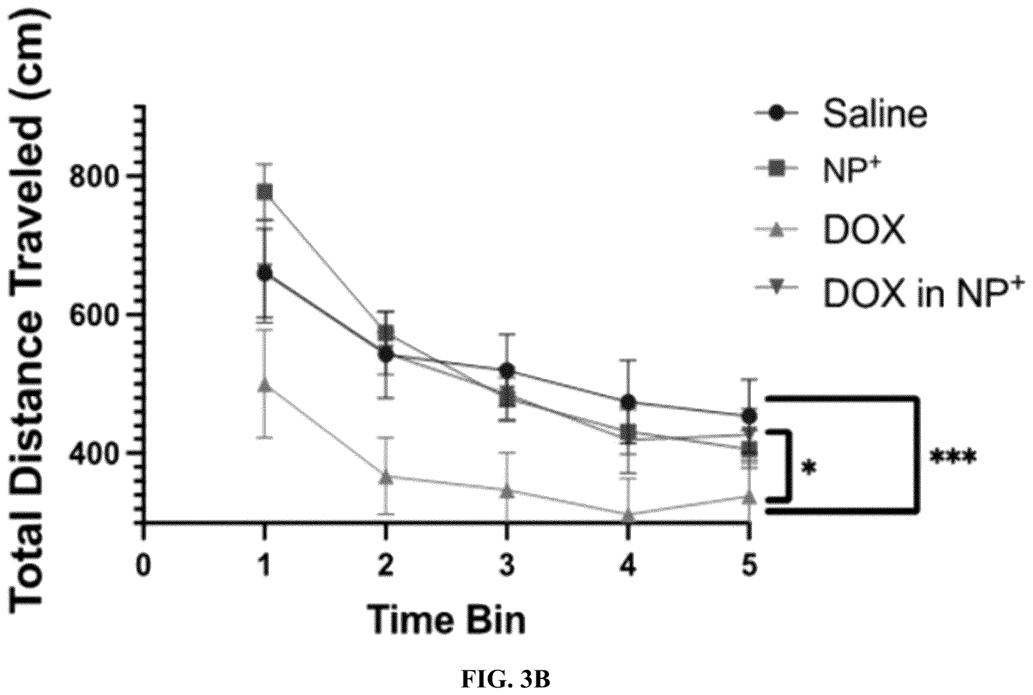

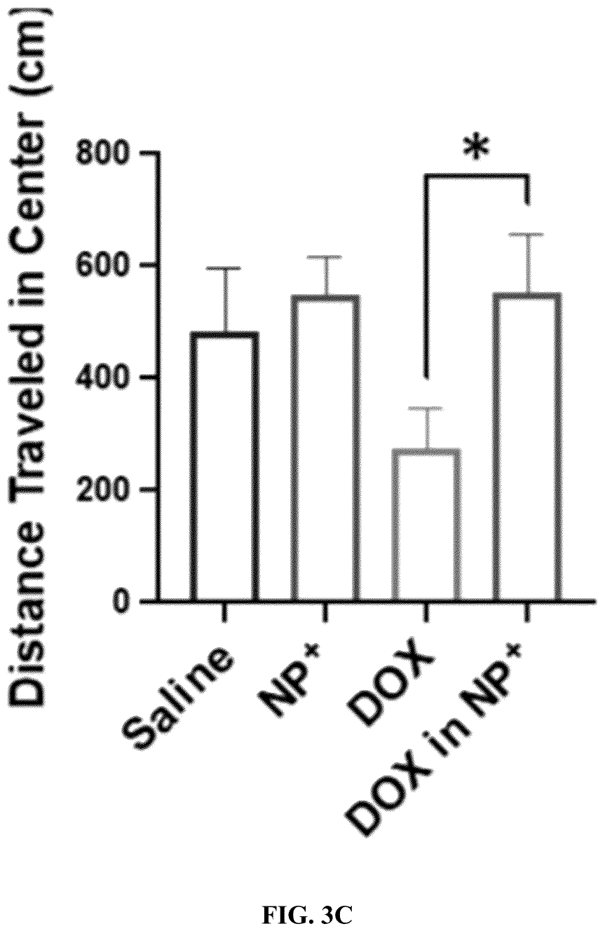

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### DETAILED DESCRIPTION

PAMAM-based nanocarriers with high loading efficiency of chemotherapeutics and high cfNA binding ability can deliver chemotherapeutics while inhibiting the systemic inflammation caused by chemotherapy. These nanocarriers can deliver chemodrugs in a sustained and local manner to increase antitumor efficacy by improving the drug pharmacokinetics. Thus, a chemotherapeutic loaded nanoparticle system can be developed to treat both primary and metastatic tumor with attenuated cognitive impairment.

The cationic polymeric nanocarriers are synthesized by modification of PAMAM dendrimers with cholesterol. The nanoparticles can be prepared through oil-in-water single emulsion with drug loading. During the emulsion process, the cholesterol modified PAMAM dendrimers can self-assemble to form nanoparticles based on their amphiphilicity. The hydrophilic PAMAM dendrimers will be the outer surface of the nanoparticle, providing positive charge environment for cfNA binding. The hydrophobic cholesterol segment will form the core of the nanoparticle and encapsulate chemotherapeutics. which formed nanoparticles with sizes of ˜160 nm and zeta potentials above +50 mV. The cfNA binding ability and drug loading efficiency of nanocarriers can be regulated by different conjugation numbers of cholesterol.

Several chemotherapy drugs have the potential to be encapsulated within polymeric nanocarriers. Non-limiting examples of chemotherapy drugs include alkylating agents such as altretamine, busulfan, carboplatin, carmustine, cisplatin, cyclophosphamide, dacarbazine, ifosfamide, lomustine, melphalan, temozolomide, or trabectedin; antimetabolites such as 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, or trifluridine and tipiracil; plant alkaloids such as vincristine, vinblastine, vinorelbine, paclitaxel, docetaxel, etoposide, teniposide, irinotecan, or topotecan; and anti-tumor antibiotics such as daunorubicin, doxorubicin, epirubicin, idarubicin, or valrubicin.

In some embodiments, the chemotherapy drug is one or both of paclitaxel or doxorubicin.

In certain embodiments, the PAMAM (polyamidoamine) dendrimers modified with cholesterol comprise at least one cholesterol residue at a terminal position of the PAMAM. In certain embodiments, the cholesterol residue is attached to an external amine functional group of the PAMAM.

In some embodiments, the nanoparticles can additionally comprise one or more immune check-point inhibitor. Immune check-point inhibitors block proteins called checkpoints that are made by some types of immune system cells, such as T cells, and some cancer cells. Such checkpoints help keep the immune response from being too strong and sometimes. Too strong a response can stop T cells from killing cancer cells. When these checkpoints are blocked, T cells can kill cancer cells more efficiently. Non-limiting examples of immune check-point inhibitors include PD-1 inhibitors such as pembrolizumab, nivolumab, or cemiplimab; PD-L1 inhibitors such as atezolizumab, avelumab, or durvalumab; CTLA-4 inhibitors such as ipilimumab or tremelimumab; and LAG-3 inhibitors such as relatlimab.

The anticancer efficacy of the nanoparticles has been evaluated in a NSG mouse model bearing MDA-MB-231 human breast tumor with lung metastasis. The paclitaxel loaded nanoparticles show higher primary tumor inhibition efficacy than the free drug, which results from the improved stability and tumor targeting efficiency. A thorough characterization of lung metastasis has been performed with a combination of IVIS imaging, H&E staining, and machine learning. The results indicate that paclitaxel free drug, although inhibits primary tumor, shows no therapeutic effect to metastasis, and even induce more metastasis. In contrast, the paclitaxel loaded nanoparticles inhibit tumor metastasis significantly. The anti-metastasis effect is correlated with serum cfDNA levels, suggesting the importance of cfNA binding against cancer metastasis.

Chemotherapy-induced cognitive impairment has been evaluated in a Balb/c mouse model treated with doxorubicin free drug and drug loaded nanoparticles. In order to detect if mice experienced any behavioral changes under treatments, open field tests and pain sensitivity behavioral tests were performed. The open field test results exhibit a significant attenuation in the distance that the doxorubicin-treated mice traveled compared to the saline control mice and the doxorubicin-loaded nanoparticle-treated mice. These results suggest that doxorubicin may induce cognitive impairment while the nanocarriers mitigate this effect. Our findings are illustrated in the figures as follows.

Several nonlimiting applications of the disclosed methods are discussed below.

A first application is to use this system to deliver chemodrug to inhibit untreated early-stage cancer and reduce the risk of recurrence after surgery/chemotherapy/radiotherapy. In cancer patients, cfNA levels are elevated in blood when compared to healthy controls. The level of cfNAs will further increase following surgery, chemotherapy, or radiation, and elevate in patients with metastatic cancer.

A second application is to use this system to deliver chemodrug to inhibit untreated metastatic cancer. Chemodrug can inhibit primary tumor growth but may increase the risk of cancer metastasis and cognitive impairment. The cationic nanocarriers will scavenge proinflammatory cfNAs to inhibit these side effects.

A third application is to use this system to treat metastatic cancers that are resistant to other treatments.

A fourth application is to use this system after surgery/chemotherapy/radiotherapy to prevent relapse.

A fifth application is to use this system to deliver immune checkpoint inhibitors and combine immune-checkpoint blockade therapy and cfNA scavenger. Delivery of immune checkpoint inhibitors to cancer will diminish anticancer immunosuppression and cfNA scavenger will attenuate pro-metastatic inflammation.

The technology will help at least three groups of patients: 1) patients with high-risk early-stage cancer, such as for example breast cancer, who need adjuvant chemotherapy to reduce the risk of recurrence after surgery; 2) patients with metastatic cancer, such as metastatic breast cancer, who need systemic chemotherapy to extend survival; 3) patients with metastatic cancer, such as metastatic breast cancer, but resistant to other treatment. All groups might benefit from our nanomedicines' ability to decrease the side effects of cytotoxic agents and prevent the development of additional tumor metastases from circulating tumor cells. The approach here has the potential to be rapidly translated in a clinical setting, as some of the materials we used are already approved for pharmacological use in human patients.

We conducted an in vivo study with Balb/c mice bearing murine 4T1 breast tumors. We demonstrated that the drug loaded scavenger nanoparticles can inhibit both primary tumor growth and cancer metastasis. We are studying how these nanoparticles modify tumor microenvironment and determining the influence of the nanoparticles on neuro-inflammation, mouse behavior (specifically anxiety), and levels of neuronal damage. We will also evaluate the side effects, biodistribution, and pharmacokinetics of the nanoparticles.

As required, detailed embodiments of the present invention are disclosed herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

The present invention may be understood more readily by reference to the following detailed description taken in connection with the accompanying figures and examples, which form a part of this disclosure. It is to be understood that this invention is not limited to the specific materials, devices, methods, applications, conditions or parameters described and/or shown herein, and that the terminology used herein is for the purpose of describing particular embodiments by way of example only and is not intended to be limiting of the claimed invention. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention, which are, for clarity, described herein in the context of separate embodiments, may also be provided in combination in a single embodiment. Conversely, various features of the invention that are, for brevity, described in the context of a single embodiment, may also be provided separately or in any subcombination. Further reference to values stated in ranges includes each and every value and combination of values within that range.

The following definitions are intended to assist in understanding the present invention.

As used herein, the singular forms “a,” “an,” and “the” include the plural, and reference to a particular numerical value includes at least that particular value, unless the context clearly dictates otherwise.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

The term “sphere-like” means an object that resembles a sphere. In some embodiments, the object is such that each point on the surface of the object is approximately the same difference from the center of the object. The approximately same difference can vary by 5%, 10%, 15% or 20% from an average difference.

1. A polymeric nanocarrier for delivery of chemotherapeutics comprising cationic polyamidoamine (PAMAM) dendrimers modified with cholesterol.

2. The polymeric nanocarrier of claim 1, additionally comprising at least one chemotherapy drug.

3. The polymeric nanocarrier of claim 2, wherein the at least one chemotherapy drug comprises at least one of paclitaxel or doxorubicin.

4. The polymeric nanocarrier of claim 1, wherein the PAMAM (polyamidoamine) dendrimers modified with cholesterol comprise at least one cholesterol residue at a terminal position of the PAMAM.

5. The polymeric nanocarrier of claim 3, wherein the PAMAM (polyamidoamine) dendrimers modified with cholesterol comprise at least one cholesterol residue at a terminal position of the PAMAM.

6. The polymeric nanocarrier of claim 5, wherein the cholesterol residue is attached to an external amine functional group of the PAMAM.

7. The polymeric nanocarrier of claim 5, wherein the polymeric nanocarrier has a sphere-like shape.

8. The polymeric nanocarrier of claim 6, wherein the polymeric nanocarrier has a sphere-like shape.

9. A method of treating cancer in a patient by administering the polymeric nanocarrier of claim 6 to the patient.

10. The method of claim 9, wherein the cancer is one or both of a primary and metastatic tumor.

11. The method of claim 10, wherein the method prevents or reduces chemotherapy-induced cognitive impairment in the patient.

12. The method of claim 11, wherein the method prevents or reduces prometastatic effects of chemotherapy in the patient.

13. The method of claim 9, wherein the cationic polyamidoamine (PAMAM) dendrimers modified with cholesterol reduce cell free nucleic acid (cfNA) levels within the patient.

14. The method of claim 12, wherein the cationic polyamidoamine (PAMAM) dendrimers modified with cholesterol reduce cell free nucleic acid (cfNA) levels within the patient.

15. The method of claim 13, wherein the method is utilized after surgery, chemotherapy or radiotherapy to prevent cancer relapse.

16. The method of claim 14, wherein the method is utilized after surgery, chemotherapy or radiotherapy to prevent cancer relapse.

17. The method of claim 15, wherein the polymeric nanocarrier delivers at least one immune check-point inhibitor to the patient.

18. The method of claim 16, wherein the polymeric nanocarrier delivers at least one immune check-point inhibitor to the patient.

19. A method of making polymeric nanocarriers for delivery of chemotherapeutics, comprising utilizing an oil-in-water emulsion process to produce a nanocarrier comprising cationic polyamidoamine (PAMAM) dendrimers modified with cholesterol and at least one chemotherapy drug.

20. The method of claim 19, wherein the at least one chemotherapy drug comprises at least one of paclitaxel or doxorubicin.
